(19) United States
(12) Patent Application Publication
(10) Pub. No.: US 2010/0086569 A1
(43) Pub. Date: Apr. 8, 2010

(54) USE OF A FIRST HOUSE DUST MITE GROUP 2 ALLERGEN FOR TREATING ALLERGY TO A SECOND HOUSE DUST MITE GROUP 2 ALLERGEN

(75) Inventor: Birthe Ross, Copenhagen (DK)

Correspondence Address: BIRCH STEWART KOLASCH & BIRCH, PO BOX 747, FALLS CHURCH, VA, 22040-0747, US

(73) Assignee: ALK- Abello A/S, Horsholm (DK)

(21) Appl. No.: 12/524,711

(22) PCT Filed: Jan. 30, 2008
(86) PCT No.: PCT/EP2008/051138

§ 371 Date: Oct. 2, 2009

Related U.S. Application Data
(60) Provisional application No. 60/898,109, filed on Jan. 30, 2007.

(30) Foreign Application Priority Data
Jan. 30, 2007 (DE) PA2007 00143

(51) Int. Cl. A61K 39/35

(52) U.S. Cl. 424/275.1

(57) ABSTRACT

The present invention relates to the use of a Der f 2 allergen composition for the manufacture of a vaccine for preventing or treating allergy to Der p 2, as well as to the use of a Der p 2 allergen composition for the manufacture of a vaccine for treating allergy to Der f 2.

### TECHNICAL FIELD

The present invention relates to the use of a Der f 2 allergen composition for the manufacture of a vaccine for preventing or treating allergy to Der p 2, and to the use of a Der p 2 allergen composition for the manufacture of a vaccine for treating allergy to Der f 2.

### BACKGROUND OF THE INVENTION

Allergy is a major health problem in countries where Western lifestyle is adapted. Furthermore, the prevalence of allergic disease is increasing in these countries. Although allergy in general may not be considered a life-threatening disease, asthma annually causes a significant number of deaths. An exceptional prevalence of about 30% in teenagers conveys a substantial loss in quality of life, working days and money, and warrants a classification among major health problems in the Western world.

Allergy is a complex disease. Many factors contribute to the sensitisation event. Among these is the susceptibility of the individual defined by an as yet insufficiently understood interplay between several genes. Another important factor is allergen exposure above certain thresholds. Several environmental factors may be important in the sensitisation process including pollution, childhood infections, parasite infections, intestinal microorganisms, etc. Once an individual is sensitised and the allergic immune response established, the presence of only minute amounts of allergen is efficiently translated into symptoms.

The natural course of allergic disease is usually accompanied by aggravation at two levels. Firstly, a progression of symptoms and disease severity, as well as disease progression, for example from hay fever to asthma. Secondly, dissemination in offending allergens most often occurs resulting in allergic multi-reactivity. Chronic inflammation leads to a general weakening of the mucosal defense mechanisms resulting in unspecific irritation and eventually destruction of the mucosal tissue. Infants may become sensitised primarily to foods, i.e. milk, resulting in eczema or gastrointestinal disorders; however, most often they outgrow these symptoms spontaneously. These infants are at risk of developing inhalation allergy later in their lives.

The most important allergen sources are found among the most prevalent particles of a certain size in the air we breathe. These sources are remarkably universal and include grass pollens and house dust mite faecal particles, which together are responsible for approximately 50% of all allergies. Of global importance are also animal dander, i.e. cat and dog dander, other pollens, such as mugwort pollens, and micro-fungi, such as Alternaria. On a regional basis yet other pollens may dominate, such as birch pollen in Northern and Central Europe, ragweed in the Eastern and Central United States, and Japanese cedar pollen in Japan. Insects, i.e. bee and wasp venoms, and foods each account for approximately 2% of all allergies.

Allergy, i.e. type I hyper-sensitivity, is caused by an inappropriate immunological reaction to foreign non-pathogenic substances. Important clinical manifestations of allergy include asthma, hay fever, eczema, and gastro intestinal disorders. The allergic reaction is prompt and peaks within 20 minutes upon contact with the offending allergen. Furthermore, the allergic reaction is specific in the sense that a particular individual is sensitised to particular allergen(s), whereas the individual does not necessarily show an allergic reaction to other substances known to cause allergic disease. The allergic phenotype is characterized by a pronounced inflammation of the mucosa of the target organ and by the presence of allergen specific antibody of the IgE class in the circulation and on the surface of mast-cells and basophils.

An allergic attack is initiated by the reaction of the foreign allergen with allergen specific IgE antibodies, when the antibodies are bound to high affinity IgE specific receptors on the surface of mast-cells and basophils. The mast-cells and basophils contain preformed mediators, i.e. histamine, tryptase, and other substances, which are released upon cross-linking of two or more receptor-bound IgE antibodies. IgE antibodies are cross-linked by the simultaneous binding of one allergen molecule. It therefore follows that a foreign substance having only one antibody binding epitope does not initiate an allergic reaction. The cross-linking of receptor bound IgE on the surface of mast-cells also leads to release of signaling molecules responsible for the attraction of eosinophils, allergen specific T-cells, and other types of cells to the site of the allergic response. These cells in interplay with allergen, IgE and effector cells, lead to a renewed flash of symptoms occurring 12-24 hours after allergen encounter (late phase reaction).

Allergy disease management comprises diagnosis and treatment including prophylactic treatments. Diagnosis of allergy is concerned with by the demonstration of allergen specific IgE and identification of the allergen source. In many cases a careful anamnesis may be sufficient for the diagnosis of allergy and for the identification of the offending allergen source material. Most often, however, the diagnosis is supported by objective measures, such as skin prick test, blood test, or provocation test.

The therapeutic options fall in three major categories. The first opportunity is allergen avoidance or reduction of the exposure. Whereas allergen avoidance is obvious e.g. in the case of food allergens, it may be difficult or expensive, as for house dust mite allergens, or it may be impossible, as for pollen allergens. The second and most widely used therapeutic option is the prescription of classical symptomatic drugs like anti-histamines and steroids. Symptomatic drugs are safe and efficient; however, they do not alter the natural cause of the disease, neither do they control the disease dissemination. The third therapeutic alternative is specific allergy vaccination that in most cases reduces or alleviates the allergic symptoms caused by the allergen in question.

Conventional specific allergy vaccination is a causal treatment for allergic disease. It interferes with basic immunological mechanisms resulting in persistent improvement of the patients' immune status. Thus, the protective effect of specific allergy vaccination extends beyond the treatment period in contrast to symptomatic drug treatment. Some patients receiving the treatment are cured, and in addition, most patients experience a relief in disease severity and symptoms experienced, or at least an arrest in disease aggravation. Thus, specific allergy vaccination has preventive effects reducing the risk of hay fever developing into asthma, and reducing the risk of developing new sensitivities.

The immunological mechanism underlying successful allergy vaccination is not known in detail. A specific immune response, such as the production of antibodies against a particular pathogen, is known as an adaptive immune response. This response can be distinguished from the innate immune response, which is an unspecific reaction towards pathogens. An allergy vaccine is bound to address the adaptive immune response, which includes cells and molecules with antigen specificity, such as T-cells and the antibody producing B-cells. B-cells cannot mature into antibody producing cells without help from T-cells of the corresponding specificity. T-cells that participate in the stimulation of allergic immune responses are primarily of the Th2 type. Establishment of a new balance between Th1 and Th2 cells has been proposed to be beneficial and central to the immunological mechanism of specific allergy vaccination. Whether this is brought about by a reduction in Th2 cells, a shift from Th2 to Th1 cells, or an up-regulation of Th1 cells is controversial. Recently, regulatory T-cells have been proposed to be important for the mechanism of allergy vaccination. According to this model regulatory T-cells, i.e. Th3 or Tr1 cells, down-regulate both Th1 and Th2 cells of the corresponding antigen specificity. In spite of these ambiguities it is generally believed that an active vaccine must have the capacity to stimulate allergen specific T-cells, preferably TH1 cells.

Specific allergy vaccination is, in spite of its virtues, not in widespread use, primarily for two reasons. One reason is the inconveniences associated with the traditional vaccination programme that comprises repeated vaccinations i.a. injections over a several months. The other reason is, more importantly, the risk of allergic side reactions. Ordinary vaccinations against infectious agents are efficiently performed using a single or a few high dose immunizations. This strategy, however, cannot be used for allergy vaccination since a pathological immune response is already ongoing.

Conventional specific allergy vaccination is therefore carried out using multiple subcutaneous immunizations applied over an extended time period. The course is divided in two phases, the up dosing and the maintenance phase. In the up dosing phase increasing doses are applied, typically over a 16-week period, starting with minute doses. When the recommended maintenance dose is reached, this dose is applied for the maintenance phase, typically with injections every six weeks. Following each injection the patient must remain under medical attendance for 30 minutes due to the risk of anaphylactic side reactions, which in principle although extremely rare could be life-threatening. In addition, the clinic should be equipped to support emergency treatment. There is no doubt that a vaccine based on a different route of administration would eliminate or reduce the risk for allergic side reactions inherent in the current subcutaneous based vaccine as well as would facilitate a more widespread use, possibly even enabling self vaccination at home.

Attempts to improve vaccines for specific allergy vaccination have been performed for over 30 years and include multifarious approaches. Several approaches have addressed the allergen itself through modification of the IgE reactivity.

Various commercial products, e.g. Alutard® SQ containing purified Der p extract or Der f extract as active substance, exist for treating allergy to the same allergen composition as that used as active substance.

Hales et al., Clinical and Experimental Allergy, 2000, Volume 30, pages 927-933, describes an experimental study showing that the proliferative and IL-5 cytokine responses to the group 1 and 7 allergens from Der p and Der f indicate that there is a large degree of T-cell cross-reactivity between the whole purified allergens from each species.

Smith et al., J Allergy Clin Immunol, 2001 June, 107(6):977-84, describes a study of the molecular basis of antigenic cross-reactivity between the group 2 mite allergens. The article e.g. discloses the fact that Der p 2 and Der f 2 have allergenic cross-reactivity as a result of conserved antigenic surface.

The object of the present invention is to provide an improved method of treating house dust mite allergy.

### SUMMARY OF THE INVENTION

This object is obtained with the present invention, which relates to the use of a Der f 2 allergen composition for the manufacture of a vaccine for preventing or treating allergy to Der p 2, and to the use of a Der p 2 allergen composition for the manufacture of a vaccine for preventing or treating allergy to Der f 2.

The present invention is based on the novel experimental finding that Der p 2 and Der f 2 have in vivo T cell cross-reactivity, i.e. experiments have shown that both rDer p 2 and rDer f 2 can stimulate the proliferation of T cells isolated from rDer f 2 sensitised mice. The invention is further based on the surprising experimental finding that Der p 2 is in fact more effective in preventing allergy to Der f 2 than Der f 2 itself, and vice versa, i.e. experiments have shown that rDer p 2 SLIT treatment followed by rDer f 2 challenges led to significant tolerance induction, in contrast to rDer f 2 SLIT treatment followed by rDer f 2 challenges, and vice versa.

Accordingly, the present invention has provided the possibility of preventing or treating allergy to both the Der p allergen specie and the Der f specie using only one of the two species, and it is possible to use either of the two species for this purpose. Furthermore, in using one species for the treatment of allergy against the other species the prophylactic or therapeutic effect against allergy to the said other specie will be increased compared to a treatment, wherein the same specie is used.

The invention further relates to a Der f 2 allergen composition for use as a vaccine for preventing or treating allergy to Der p 2. Likewise, the invention relates to a Der p 2 allergen composition for use as a vaccine for treating allergy to Der f 2.

The invention further relates to a method of preventing or treating allergy to Der p 2 comprising the step of administering a vaccine comprising a Der f 2 composition to a subject. Likewise, the invention relates to a method of preventing or treating allergy to Der f 2 comprising the step of administering a vaccine comprising a Der p 2 composition to a subject.

### SHORT DESCRIPTION OF THE FIGURES

FIG. 1A shows the T cell response for mice i.p. immunised twice with rDer f 2 and in vitro re-stimulated with either rDer f 2 or rDer p 2.

FIG. 1B shows the T cell response for mice i.p. immunised three times with rDer f 2 and in vitro re-stimulated with either rDer f 2 or rDer p 2.

FIG. 2A shows the timeline for the treatment protocol of mouse model experiments investigating the effect of SLIT treatment in tolerance induction.

FIG. 2B shows the T cell response for mice subjected to SLIT treatment with either rDer f 2 or rDer p 2 followed by rDer f 2 challenges.

FIG. 2C shows the T cell response for mice subjected to SLIT treatment with either rDer f 2 or rDer p 2 followed by rDer p 2 challenges.

### DETAILED DESCRIPTION OF THE INVENTION

### Der f 2 and Der p 2 Composition

The allergen composition of the invention may be in the form of an allergen extract, a purified fraction of an allergen extract, a modified allergen, a recombinant allergen or a mutant of a recombinant allergen. An allergenic extract may naturally contain one or more isoforms of the same allergen, whereas a recombinant allergen typically only represents one isoform of an allergen. The mutant allergen may be a low IgE-binding mutant, e.g. a low IgE-binding allergen according to WO 99/47680, WO 02/40676 or WO 03/096869 A2. In a preferred embodiment of the invention, the allergen composition is an allergen extract, a purified fraction of an allergen extract or a recombinant allergen.

Allergens may be present in equi-molar amounts or the ratio of the allergens present may vary preferably up to 1:20.

### Preventing and Treating Allergy

Specific allergy vaccination (SAV), formerly known as Specific Immunotheraphy or Hyposensitization, has been used for the treatment of Type 1 IgE mediated allergic disease since the beginning of this century.

The general benefits obtained through SAV are: a) reduction of allergic symptoms and medicine consumption, b) improved tolerance towards the allergens in the eyes, nose and lungs and c) reduced skin reactivity (early and late phase reactions).

The basic mechanism behind the improvement obtained by SAV is unknown, but a number of common features can be extracted from the numerous SAV studies performed in the last decades: 1) the amount of total IgE is unchanged during the treatment period, 2) the amount of allergen specific IgE increases transiently during updosing, then it falls back to the initial (pretreatment) level, 3) the epitope specificity and affinity of IgE remains unchanged, 4) allergen specific IgG, in particularly IgG4, raises sharply during SAV, 5) a new Th0/1/Reg response is apparently initiated and 6) the Th2 response seem unchanged. There is no correlation between the effect induced by SAV and the onset of specific IgG.

SAV induces a new immune response which matures during the treatment period (Th0/1 T-cells are recruited, an allergen specific IgX (X may be A1, A2, G1, G2, G3, G4, M or D) is initiated). As the affinity (or amount/affinity) of the new antibody response, IgX, has matured, IgX may compete efficiently with IgE for the allergen(s), inhibiting the “normal” Th2 based allergic response characterised by the cross-linking of receptor bound IgE on the surface of mast-cells and basophils. Hence, clinical symptoms will gradually be reduced.

It is believed that the preventive treatment carried out in the present invention at least partly functions by way of the same mechanisms as disclosed above for SAV.

In one embodiment of the invention, the vaccine is for preventing or treating allergy in a subject unsensitised to the allergen. In another embodiment of the invention, the vaccine is for preventing or treating allergy in a subject sensitised to the allergen.

In many geographical regions both of the Der p and Der f house dust mite species are present in the environment. Accordingly, the population consists of a subpopulation, which is sensitised to both species, a subpopulation which is sensitised to one of the two species and unsensitised to the other species and a subpopulation which is unsensitised to both species. Individuals, who are unsensitised to one or both of the two species are of course likely to become sensitised in time. The fact that a subject is sensitised does not necessarily mean that the subject has any clinical symptoms of allergy yet, but there is a risk that such clinical symptoms may develop in time. Thus, when both species are present in the environment prophylactic or therapeutic treatment against allergy to both species are relevant and desirable. In accordance with the present invention such treatment against allergy to both species can be achieved with either one of the two species.

When only one of the Der p and Der f house dust mite species is present in the environment, it is according to the present invention advantageous to use the other species for the prophylactic or therapeutic treatment.

### Vaccine Formulation

The vaccine of the present invention may be any conventional vaccine formulation, including vaccines suitable for parenteral or mucosal administration.

### Parenteral Administration

In one embodiment of the invention the treatment is carried out by parenteral administration. Parenteral administration includes intravenous, intramuscular, intraarticular, subcutaneous, intradermal, epicutaneous/transdermal and intraperitoneal administration. Vaccines for administration via injection may be formulated so as to be suitable for injection by needle or for needleless injection.

Preparation of vaccines is generally well known in the art. The allergen may suitably be mixed with excipients which are pharmaceutically acceptable and further compatible with the active ingredient. Examples of suitable excipients are water, saline, dextrose, glycerol, ethanol and the like as well as combinations thereof. The vaccine may additionally contain other substances such as wetting agents, emulsifying agents, buffering agents or adjuvants enhancing the effectiveness of the vaccine.

Vaccines may suitably be formulated with excipients normally employed for such formulations, e.g. pharmaceutical grades of mannitol, lactose, starch, magnesium stearate, sodium saccharine, cellulose, magnesium carbonate and the like.

The vaccines are administered in a way so as to be compatible with the dosage formulation and in such amount as will be therapeutically effective and immunogenic. The quantity of active component contained within the vaccine depends on the subject to be treated, i.a. the capability of the subjects immune system to respond to the treatment, the route of administration and the age and weight of the subject. In general the treatment may e.g. consist in a treatment protocol, which comprises an up-dosing period, during which the dose is slowly raised, and a maintenance period, wherein the patient receives a fixed maintenance dose. Up-dosing may e.g. comprise 10-20 administrations, carried out weekly or biweekly. In the maintenance dose the patient is treated e.g. monthly or bi-monthly for a period of up to three years. This is contemplated to give desired level of prophylactic or therapeutic effect.

In the case of parenteral administration by e.g. subcutaneous injections, the treatment comprises at least one administration, preferably 1-40 administrations. Suitable dosage ranges can vary within the range from about 0.0001 μg to 1000 μg. Expressed as SQ-u the doses may vary from 20 SQ-u to 100000 SQ-u.

In a particular embodiment of the method of the invention, one or more additional rounds of treatment are carried out subsequent to the end of a first treatment round to re-stimulate (boost) the immune system further. Such additional rounds of treatment may e.g. involve a limited number of administrations, e.g. from 1-10, preferably 1-5, over a period of e.g. from one to four weeks. Patients may e.g. be subjected to such additional rounds of treatment one or two times each year. Such a treatment protocol has the advantage of being very effective while at the same time limiting the number of parenteral administrations to a minimum. It is desired to reduce the number of parenteral administrations to a minimum, since such administrations should be performed by specialists and further require post-administration attendance for a period of time.

### Mucosal Administration

The mucosa to which the allergy vaccine is administered may be any suitable mucosa, and the administration includes oral (via the mucosa of the digestive system), nasal, vaginal, sublingual, ocular, rectal, urinal, intramammal, pulmonal, otolar (i.e. via the ear) and buccal administration, preferably buccal or sublingual administration (oromucosal administration). The allergy vaccine may be in the form of a spray, an aerosol, a mixture, a suspension, a dispersion, an emulsion, a gel, a paste, a syrup, a cream, an ointment, implants (ear, eye, skin, nose, rectal, and vaginal), intramammary preparations, vagitories, suppositories, or uteritories.

It has been speculated that it is preferable to carry out a mucosal administration of a vaccine via the mucosa, which is subject to the natural exposure to the allergen. Accordingly, for allergies to airborne mucosal antigenic agents, it is preferred to use administration via the respiratory system, preferably an oromucosal administration.

In one embodiment of the invention, the subject is subjected to a vaccination protocol comprising daily administration of the vaccine. In another embodiment of the invention the vaccination protocol comprises administration of the vaccine every second day, every third day or every fourth day. For instance, the vaccination protocol comprises administration of the vaccine for a period of more than 4 weeks, preferably more than 8 weeks, more preferably more than 12 weeks, more preferably more than 16 weeks, more preferably more than 20 weeks, more preferably more than 24 weeks, more preferably more than 30 and most preferably more than 36 weeks.

The period of administration may a continuous period. Alternatively, the period of administration is a discontinuous period interrupted by one or more periods of non-administration. Preferably, the (total) period of non-administration is shorter than the (total) period of administration.

In a further embodiment of the invention, the vaccine is administered to the patient once a day. Alternatively, the vaccine is administered to the patient twice a day. The vaccine may be a uni-dose vaccine.

### Oromucosal Administration

The oromucosal administration may be carried out using any available oromucosal administration formulation, including a solution, a suspension, fast dispersing dosage forms, drops and lozenges.

In a preferred embodiment of the invention, sublingual immunotherapy (SLIT) is used, in which case fast dispersing dosage forms, drops and lozenges are preferred formulations.

Examples of fast dispersing dosage forms are those disclosed in U.S. Pat. No. 5,648,093, WO 00/51568, WO 02/13858, WO99/21579, WO 00/44351, U.S. Pat. No. 4,371,516 and EP-278 877, as well as WO 2004/047794 and WO 2004/075875 filed in the assignee name of ALK-Abelló A/S. Preferred fast dispersing dosage forms are those produced by freeze-drying. Preferred matrix forming agents are fish gelatine and modified starch.

Allergy vaccines in the form of an aqueous solution or a fast-dispersing tablet, cf. WO 04/047794, are particularly suitable for buccal and sublingual administration.

Classical incremental dosage desensitisation, where the dose of allergen in the form of a fast dispersing solid dosage form is increased to a certain maximum, may be used in the present invention. The preferred potency of a unit dose of the dosage form is from 150-1000000 SQ-u/dosage form, more preferred the potency is from 500-500000 SQ-u/dosage form and more preferably the potency is from 1000-250000 SQ-u/dosage form, even more preferred 1500-125000 SQ-u/dosage form most preferable 1500-75000 SQ-u/dosage form.

In another embodiment of the invention the dosage form is a repeated mono-dose, preferably within the range of 1500-75000 SQ-u/dosage form.

The allergy vaccine used in the method of the invention may be in the form of any formulation suitable for administration to a mucosal surface, including a spray, an aerosol, a mixture, tablets (entero- and not-enterocoated), capsule (hard and soft, entero- and not-enterocoated), a suspension, a dispersion, granules, a powder, a solution, an emulsion, chewable tablets, drops, a gel, a paste, a syrup, a cream, a losenge (powder, granulate, tablets), a fast-dispersing tablet, an instillation fluid, a gas, a vapour, an ointment, a stick, implants (ear, eye, skin, nose, rectal, and vaginal), intramammary preparations, vagitories, suppositories, or uteritories.

It is to be understood that the vaccine of the invention may further comprise additional adjuvants and other excipients suitable for such type of formulation. Such additional adjuvants and excipients are well-known to the person skilled in the art and include i.a. solvents, emulsifiers, wetting agents, plasticizers, colouring substances, fillers, preservatives, viscosity adjusting agents, buffering agents, mucoadhesive substances, and the like. Examples of formulation strategies are well-known to the person skilled in the art.

### Adjuvant

The allergy vaccine may include an adjuvant, which may be any conventional adjuvant, including oxygen-containing metal salts, heat-labile enterotoxin (LT), cholera toxin (CT), cholera toxin B subunit (CTB), polymerised liposomes, mutant toxins, e.g. LTK63 and LTR72, microcapsules, interleukins (e.g. IL-1β, IL-2, IL-7, IL-12, INFγ), GM-CSF, MDF derivatives, CpG oligonucleotides, LPS, MPL, phosphophazenes, Adju-Phos®, glucan, antigen formulation, liposomes, DDE, DHEA, DMPC, DMPG, DOC/Alum Complex, Freund's incomplete adjuvant, ISCOMs®, LT Oral Adjuvant, muramyl dipeptide, monophosphoryl lipid A, muramyl tripeptide, and phosphatidylethanolamine.

The oxygen-containing metal salt may be any oxygen-containing metal salt providing the desired effect. In a preferred embodiment, the cation of the oxygen-containing metal salt is selected from Al, K, Ca, Mg, Zn, Ba, Na, Li, B, Be, Fe, Si, Co, Cu, Ni, Ag, Au, and Cr. In a preferred embodiment, the anion of the oxygen-containing metal salt is selected from sulphates, hydroxides, phosphates, nitrates, iodates, bromates, carbonates, hydrates, acetates, citrates, oxalates, and tartrates, and mixed forms thereof. Examples are aluminium hydroxide, aluminium phosphate, aluminium sulphate, potassium aluminium sulphate, calcium phosphate, Maalox (mixture of aluminium hydroxide and magnesium hydroxide), beryllium hydroxide, zinc hydroxide, zinc carbonate, zinc chloride, and barium sulphate.

### DEFINITIONS

In connection with the present invention the following definitions are used:

The term “Der f” means the house dust mite species called Dermaphagoides farinae. The term “Der f 2” means the Der f allergen belonging to the group 2 according to the allergen nomenclature of the Allergen Nomenclature Sub-committee, which operates under the auspices of the International Union of Immunological Societies (I.U.I.S.) and the World Health Organisation (W.H.O.).

The term “Der p” means the house dust mite species called Dermaphagoides pteronyssinus. The term “Der p 2” means the Der f allergen belonging to the group 2 according to the allergen nomenclature of the Allergen Nomenclature Sub-committee, which operates under the auspices of the International Union of Immunological Societies (I.U.I.S.) and the World Health Organisation (W.H.O.).

The term “treating” means partly or wholly curing or alleviating symptoms, or inhibiting causes of symptoms.

The term “preventing” means any type of prophylactic treatment, including partly or wholly preventing or inhibiting the development of symptoms or the development of causes of symptoms.

The term “oromucosal administration” refers to a route of administration where the dosage form is placed under the tongue or anywhere else in the oral cavity (buccal administration) to allow the active ingredient to come in contact with the mucosa of the oral cavity or the pharynx of the patient in order to obtain a local or systemic effect of the active ingredient. An example of an oromucosal administration route is sublingual administration.

The term “sublingual administration” refers to a route of administration, where a dosage form is placed underneath the tongue in order to obtain a local or systemic effect of the active ingredient.

The term “SQ-u” means SQ-Unit: The SQ-Unit is determined in accordance with ALK-Abelló A/S's “SQ biopotency”-standardisation method, where 100,000 SQ units equal the standard subcutaneous maintenance dose. Normally 1 mg of extract contains between 100,000 and 1,000,000 SQ-Units, depending on the allergen source from which they originate and the manufacturing process used. The precise allergen amount can be determined by means of immunoassay i.e. total major allergen content and total allergen activity.

The term “allergy” means any type of hypersensitivity reaction to an environmental allergen mediated by immunological mechanisms, including Type I-IV hypersensitivity reactions, including allergic rhinitis, asthma and atopic dermatitis.

The term “unsensitised” means that the subject to be treated exhibits no IgE response specific to the allergen administered. In connection with the present invention the expression “exhibits no IgE response specific to the allergen” means a level of allergen-specific IgE antibody undetectable in a conventional immunoassay.

The expression “allergy to Der p 2” means allergy to environmental Der p as such or any other composition containing Der p 2.

The expression “allergy to Der f 2” means allergy to environmental Der f as such or any other composition containing Der f 2.

### EXAMPLES

### Example 1

### SLIT Treatment with Recombinant Der f 2 and Der p 2

### Methods and Materials:

Expression and Purification of rDer f 2 and rDer p 2 from E. coli

cDNA encoding Der f 2 and Der p 2 was amplified with PCR using the primer derp2DOndeI: gcgcgccatatggatcaagtcgatgtcaaag, derp2UPxhoI: gcgcgcctcgag ttaatcgcggattttagcatg, derf2DOndeI: gcgcgccatatg gatcaggtcgatgtcaaag, derf2UPxho1: gcgcgcctcgag ttaatcgcggattttagcgtg, and the plasmids carrying the Der f 2 and Der p 2 cDNA (pCo06 and pCo10) as template. The PCR products were cloned into the pETDuet-1 vector which resulted in an extra N-terminal methionine. The vector was introduced into the Escherichia coli strain BL21(DE3). Expression of the rDer f 2 and rDer p 2 protein was induced by the addition of 1 mM isopropyl-β-thiogalactopyranoside (IPTG) by over night incubation at 37° C. Purified inclusion bodies were solubilized in 8 M urea, 20 mM Tris pH 8.5 by over night incubation. The protein was refolded by rapidly dilution to a final urea concentration of 0.65 M and one hour incubation at room temperature. Precipitated protein was removed by centrifugation. Refolded protein was purified on a 5 ml HiTrapQ HP column (Amersham Biosciences) in 20 mM Tris pH 8.5. Correctly folded protein was eluded at 90 mM NaCl. The rDer f 2 and rDer p 2 were further purified on a Superdex 75 HiLoad 16/60 column using 10 mM NH4HCO3. The purified proteins were freezedried.

### Allergen Extracts

The Der f 1:Der f 2 ratio in the Der f allergen extract is 1:0.45, while the Der p 1:Der p 2 ratio in the Der p allergens extract is 1:1.22. Der f and Der p extract concentrations are given according to their Der f 2 or Der p 2 contents, and not as total extract protein concentrations.

### Circular Dichroism (CD)

CD spectra were measured on an OLIS DSM CD-spectrophotometer (Bogart, Ga., USA) using 0.1 cm square quartz cuvettes. Spectra were recorded at 15° C. over 190-265 nm. The concentration of samples used was approximately 300 μg/ml in 10 mM sodium phosphate pH 7.0.

### Mice

6-8 week-old female SJL mice were purchased from Taconic. The mice were housed under a 12-h light, 12-h dark cycle in a specific pathogen-free environment. All experiments described in this report were conducted in accordance with Danish legislation.

### Immunizations

Groups of mice were immunized with up to three intraperitoneal injections of recombinant allergens adsorbed to aluminium hydroxide. The mice were killed on day 10-12 after the last immunization and blood was collected and serum prepared.

### SLIT Treatment

The mice were SLIT treated with a daily dose of one or several of the following substances: rDer f 2, rDer p 2 or buffer, 5 days a week for 2-4 weeks, as indicated in the results section.

### T-Cell Proliferation and Cytokine Production

The spleen was teased into single cell suspension in RPMI-1640 (BioWhittaker, Belgium) and washed three times in RPMI-1640. 3×105 cells in RPMI-1640 containing 50 g/Ml gentamicin (Gibco, UK), 1% Nutridoma (Roche, Germany) 1.5 mM monothioglycerol (Sigma) and 1% Fetal Calf Serum (Gibco, UK) were added to each well of a 96 well flat-bottomed culture plate (Nunc) and the cells were stimulated by rDer p or rDer f allergens at various concentrations. The cells were cultured at 37° C. and 5% CO2. Proliferation was measured by adding 0.5 μCi of 3H-thymidine to each well for another 18 hours, followed by harvesting the cells on a Tomtec 96 well plate harvester (Tomtec, USA) and counting the incorporated radiolabel using a Wallac Microbeta 1450 Liquid scintillation counter (Wallac, Finland).

### Statistical Analysis

Differences between experimental groups were assessed by the unpaired Mann-Whitney test. Probabilities less than or equal to 0.05 using the GraphPad Prism (Graphpad Software, Calif., USA) were considered significant.

### Antibodies

The levels of Der f or Der p-specific serum IgG1, IgG2a, IgG2b, IgE and IgA were measured on the ADVIA Centauer platform (Bayer Diagnostics, Tarrytown, N.Y.). Goat anti-mouse Ig (Southern Biotechnology, Birmingham, Ala.) covalently coupled to paramagnetic particles was used to capture the different serum Ig-isotypes. Bound solid phase Ig's were allowed to react with liquid phase purified biotinylated Der f or Der p extracts, or recombinant Der f 2 or Der p 2, which were detected as chemiluminescence using acridiniumester labelled streptavidin.

### Results and Discussion for Purification and Structure:

Purification of rDer f 2 and rDer p 2

An E. coli Der f 2 and Der p 2 expression system was used. E. coli is capable of producing high yields of protein and the initial expression of recombinant protein was very high with an approximate yield of 100 mg inclusion bodies per liter cell culture. However, upon refolding, >90% of the protein precipitated. The precipitated protein was removed by centrifugation. The remaining incorrectly folded protein was separated from the correctly folded protein by anion chromatography. The refolded and purified rDer f 2 and rDer p from E. coli gave a nice single band on a SDS-PAGE. The protein folding was verified by CD spectroscopy. CD spectra of purified natural Der f 2 and Der p 2 were compared to CD spectra of the purified refolded recombinant Der f 2 and Der p 2. The CD spectra showed that the natural and the recombinant allergens have the maximum and minimum which characterize a β-strand protein. The curves are slightly different for the natural and recombinant allergens. Since the recombinant allergens were refolded, it is likely that a minor part of the protein was not correctly folded. Furthermore, the molecular weights of the allergens were confirmed by mass spectrometry and the overall fold of the proteins was tested by the ability of the proteins to bind human mite specific IgE (RIE).

### Results for T Cell Response and Induction of Tolerance by SLIT Treatment:

### Introduction

The group 1 and group 2 allergens have been shown to sensitize more than 80% of patients with house dust mite allergy. Several recombinant allergens have been cloned and characterized, including Der f 2 and Der p 2, which have been shown to exhibit allergic activity like the native allergens. In contrast to house dust mite extracts, recombinant allergens contain a well-defined amount of allergen without additional allergens and substances such as LPS. Therefore, the recombinant single allergens can be useful in the diagnosis and immunotherapy of allergic diseases caused by house dust mites. Mouse models are important in the study of the utility of recombinant allergens in immunotherapy, for example to determine the range of different allergens that are needed to induce tolerance to the extracts. Recombinant mite allergens are also very useful when studying the cross-reactivity of mite allergens, since the allergens are not co-administered with other allergens and substances. Here, it is investigated whether it is possible to establish a murine oral tolerance model using recombinant Der f 2 in order to investigate the T cell cross-reactivity of rDer f 2 and rDer p 2. Furthermore, the effect of SLIT treatment with the recombinant allergens Der f 2 and Der p 2 on the T cell response is studied.

T Cell Response to Immunization with Recombinant Der f 2

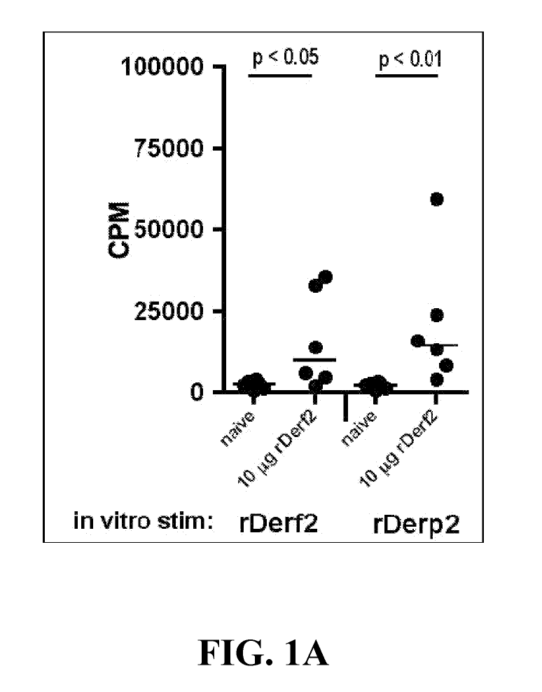

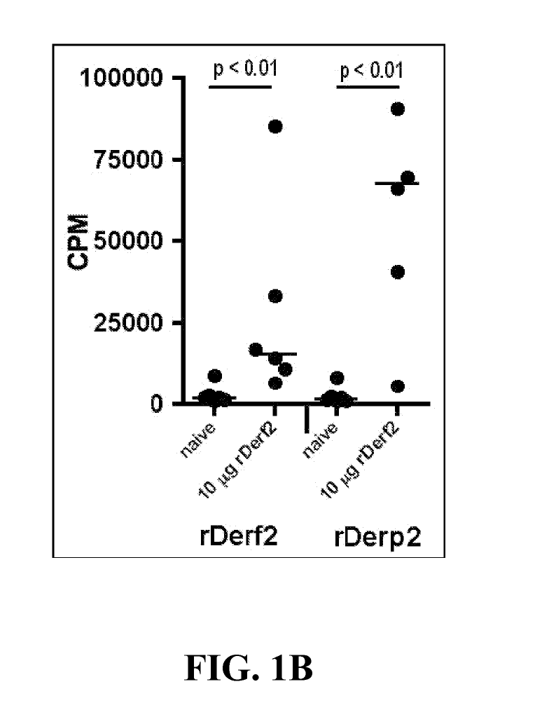

The immunization effect of rDer f 2 on the T cell response was investigated. SJL mice were i.p. immunized two and three times, respectively, with 10 μg rDer f 2 and subsequently re-stimulated in vitro with 10 μg/ml recombinant Der f 2 or Der p 2. The recombinant Der f 2 induces a T cell response that is significantly higher when immunized twice (FIG. 1A) and three times (FIG. 1B) compared to the naive mice. Very low levels of allergen specific serum IgE, IgG1 and IgG2a following the immunizations were observed (data not shown). Based on these results, a protocol using three i.p. challenges with recombinant allergens following SLIT treatment was chosen.

In order to study T cell cross-reactivity between rDer f 2 and rDer p 2, the T cells were re-stimulated with both allergens (FIG. 1). Both rDer f 2 and rDer p 2 can stimulate the proliferation of T cells from the rDer f 2 sensitized mice. This demonstrates the in vivo T cell cross-reactivity between rDer f 2 and rDer p 2.

### SLIT Treatment Induces Tolerance in Der f 2 Challenged SJL Mice

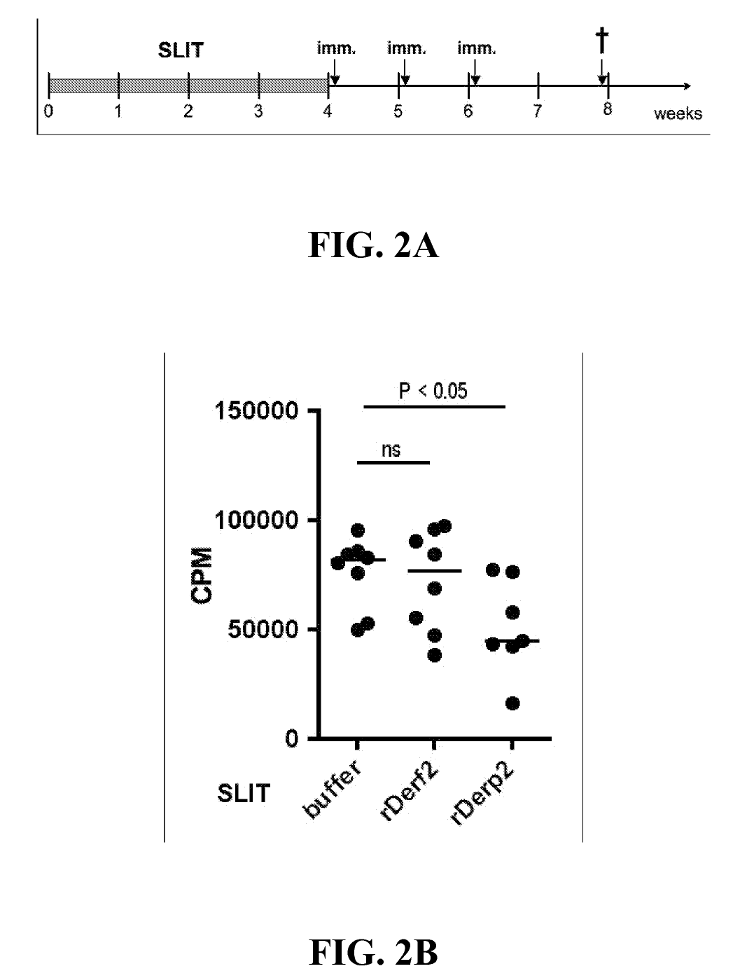

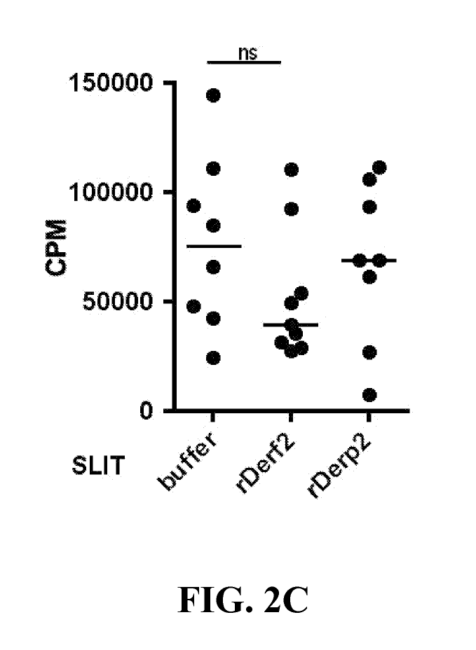

After having established the immunization schedule, the effect of SLIT treatment in SJL mice was investigated. SJL mice were treated sublingually with 10 μg rDer f 2, rDer p 2 or buffer five days a week for four weeks. This was followed by i.p. challenges with rDer f 2 or rDer p 2 (FIG. 2A). Ten to twelve days after the last challenge, allergen specific in vitro T cell proliferation of spleen cells was determined and serum was collected for determination of specific antibody levels. In addition, blood samples were collected after SLIT but before the first challenge for serum antibody analysis.

SLIT treatment with rDer f 2 followed by rDer f 2 challenges (FIG. 2B) or rDer p 2 SLIT treatment with subsequent rDer p 2 challenges (FIG. 2C) seems not to have an effect on T-cell proliferation compared to the buffer treated groups. However, rDer p 2 SLIT treatment of rDer f 2 challenged mice leads to significant tolerance induction compared to the buffer treated group (FIG. 2B). Similar to this, SLIT treatment with rDer f 2 followed by rDer p 2 challenge led to a reduced T cell response compared to the buffer treated mice group (FIG. 2C), however, not significantly. This indicates that SLIT treatment does not have an effect on the T cell response in the case where the treatment is performed using the same allergen as for the challenges, but seems to have an effect when the SLIT treatment is carried out with the corresponding allergen from the other mite species. The antibody levels were similar and low for all groups between SLIT and first challenge, and after three challenges.

### Discussion:

In the present study, oral tolerance is studied in naive animals mainly to investigate the effect on the T cell response. During the setup of the challenge schedule, T cell cross-reactivity between rDer f 2 and rDer p 2 was observed.

rDer p 2 SLIT treatment followed by rDer f 2 challenges led to significant tolerance induction, in contrast to rDer f 2 SLIT treatment. Further, in the opposite experiment of rDer f 2 SLIT treatment followed by rDer p 2 challenges, there is a non-significant tendency to a down-regulation of the T cell response.

It is speculated that the inverse relationship of the effect of the rDer f 2-rDer p 2 SLIT treatment may be the result of a sub-optimal stimulation of the T cells. The amino acid sequences of rDer f 2 and rDer p 2 differ in 15 amino acids, which are distributed over the whole sequence. Thus, the resulting T cell ligands from Der f 2 will therefore be a mix of peptides, a portion of which have one or more differences in its amino acid sequence compared to a mix of peptides from Der p 2. It is speculated that such variant peptides may give rise to a sub-optimal stimulation of the T cells.

The fact that a recombinant allergen is able to induce T cell tolerance shows that the House Dust Mite Group 2 allergens in isolated form have therapeutical potential. Further studies are needed to confirm that recombinant mite allergens can induce tolerance towards mite extracts, which contain multiple allergens. Also studies using higher doses of recombinant allergen for a longer period as well as SLIT treatment with multiple allergens will give more insight into the recombinant allergens as potential candidates for SLIT. The recombinant group 2 house dust mite allergens Der f 2 or Der p 2 as well as other recombinant house dust mite allergens could prove to be excellent alternatives to the mite extracts in SLIT treatment.

### CONCLUSION

A murine oral tolerance model using Der f and Der p extract, and recombinant Der f 2 was established. SLIT treatment with the single allergens rDer f 2 and rDer p 2 in rDer f 2 and rDer p 2 challenged mice did not seem to induce tolerance towards the same allergen. Instead the rDer p 2 allergen induced tolerance towards rDer f 2, and rDer p 2 seemed to induce tolerance towards rDer f 2.

1.-2. (canceled)

3. The method according to claim 19 or 20, wherein the allergen composition is an allergen extract, a purified fraction of an allergen extract or a recombinant allergen.

4. The method according to claim 19 or 20, wherein the vaccine is suitable for parenteral or mucosal administration.

5. The method according to claim 4, wherein the vaccine is suitable for mucosal administration.

6. The method according to claim 5, wherein the vaccine is suitable for oromucosal administration.

7. The method according to claim 6, wherein the vaccine is suitable for sublingual administration.

8. The method according to claim 19 or 20, wherein the vaccine is for preventing or treating allergy in a subject unsensitised to the allergen

9. The method according to claim 19 or 20, wherein the vaccine is for preventing or treating allergy in a subject sensitized to the allergen.

10. A Der f 2 allergen composition for use as a vaccine for preventing or treating allergy to Der p 2.

11. A Der p 2 allergen composition for use as a vaccine for treating allergy to Der f 2.

12. A composition according to claim 10 or 11, wherein the allergen composition is an allergen extract, a purified fraction of an allergen extract or a recombinant allergen.

13. A composition according to claim 10 or 11, wherein the vaccine is suitable for parenteral or mucosal administration.

14. A composition according to claim 13, wherein the vaccine is suitable for mucosal administration.

15. A composition according to claim 14, wherein the vaccine is suitable for oromucosal administration.

16. A composition according to claim 15, wherein the vaccine is suitable for sublingual administration.

17. A composition according to claim 10 or 11 for preventing or treating allergy in a subject unsensitised to the allergen.

18. A composition according to claim 10 or 11 for preventing or treating allergy in a subject sensitized to the allergen.

19. A method of preventing or treating allergy to Der p 2 comprising the step of administering a vaccine comprising a Der f 2 composition to a subject.

20. A method of preventing or treating allergy to Der f 2 comprising the step of administering a vaccine comprising a Der p 2 composition to a subject.


SEQUENCE LISTING (4 sequences)

SEQ ID NO: 1 - DNA, 31 bp, Artificial sequence
  Synthetic PCR Primer
gcgcgccatatggatcaagtcgatgtcaaag

SEQ ID NO: 2 - DNA, 33 bp, Artificial sequence
  Synthetic PCR primer
gcgcgcctcgagttaatcgcggattttagcatg

SEQ ID NO: 3 - DNA, 31 bp, Artificial sequence
  Synthetic PCR primer
gcgcgccatatggatcaggtcgatgtcaaag

SEQ ID NO: 4 - DNA, 33 bp, Artificial sequence
  Synthetic PCR primer
gcgcgcctcgagttaatcgcggattttagcgtg